(19) United States
(12) Patent Application Publication
(10) Pub. No.: US 2015/0057570 A1
(43) Pub. Date: Feb. 26, 2015

(54) DEVICES AND METHODS FOR OBTAINING TISSUE SAMPLES

(71) Applicant: Spine View, Inc., San Jose, CA (US)

(72) Inventors: Singfatt Chin, Pleasanton, CA (US); Murali Dharan, Fremont, CA (US); Jefferey Wayne Etter, Hayward, CA (US)

(73) Assignee: SPINE VIEW, INC., San Jose, CA (US)

(21) Appl. No.: 14/290,929

(22) Filed: May 29, 2014

Related U.S. Application Data
(60) Provisional application No. 61/828,649, filed on May 29, 2013.

(51) Int. Cl. A61B 19/00; A61B 18/12; A61B 10/04; A61B 18/14; A61B 10/02

(52) U.S. Cl. CPC A61B 19/201; A61B 18/1482; A61B 10/0283; A61B 10/04; A61B 18/1206; A61B 2010/045; A61B 2018/00577
USPC 600/566; 606/130; 600/567; 606/46; 600/565; 606/39

(57) ABSTRACT

Medical devices and systems, and methods of their use, are disclosed having configurations suitable for obtaining biological tissue samples suitable for analysis, such as biopsy, while minimizing undesirable collateral damage to surrounding tissue or minimizing air leaks. Certain disclosed medical systems provide for obtaining biological tissue samples, while preserving organ functionality.

This application claims priority to U.S. Provisional Application Ser. No. 61/828,649, entitled “Devices and Methods for Obtaining Tissue Samples,” filed May 29, 2013, which application is incorporated herein by reference in its entirety.

### BACKGROUND

1. Field of this Disclosure

This disclosure relates generally to medical devices, and more particularly, to medical devices employed to obtain biological tissue samples for analysis.

2. Description of the Related Art

Tissue samples of certain target tissues are desirable for analysis, in a biopsy procedure for example. Existing technologies often fail to acquire a desired volume of tissue sample and often provide undesirable damage to the tissue sample itself during extraction making the analysis thereof more difficult. Existing technologies may also fail to properly seal the tissue near to or surrounding the target tissue, resulting in undesirable bleeding or undesirable dislodging of certain target tissues. Moreover, such existing technologies may include surfaces and structures that may cause collateral damage to surrounding tissues during tissue sample or medical device extraction. Certain existing technologies are constructed from materials that are not compatible with certain viewing technologies. Such viewing technologies, for illustration purposes only, may include magnetic resonance imaging (MRI) systems, fluoroscopic imaging systems or Computed Tomography (CT) imaging systems, (collectively referred to as “Viewing Technologies”) which may lead to failed tissue acquisition procedures. Such system design faults often require additional tissue sample acquisition procedures, resulting in additional costs and further undesirable tissue damage.

What is needed is a medical system configured to properly position a medical device for tissue sample extraction. What is further needed is a medical system configured to extract a desired tissue sample of sufficient volume for analysis, preserving the tissue sample to enhance analysis thereof, and further treating the target tissue site to prevent additional undesirable tissue damage and/or minimize air leaks. Still, what is needed is a medical system constructed from materials compatible with fluoroscopic or CT viewing systems.

### BRIEF SUMMARY

Consistent with the present disclosure, medical devices and systems, and methods of their use, are disclosed having configurations suitable for obtaining biological tissue samples for analysis, such as biopsy, while preserving organ functionality and minimizing undesirable collateral damage to surrounding tissue. In a first aspect, a stabilization device includes a footer element and a directional element. The footer element includes distal and proximal ends, the distal end configured to engage a tissue surface, the proximal end including a socket portion. The directional element includes distal and proximal ends, and a lumen therethrough. The lumen of the directional element includes a central axis, and the distal end of the directional element may be configured to be operably coupled with the socket of the proximal end of the footer element. In certain embodiments, the footer element further includes a control knob. Operation of the control knob of the footer element results in fixedly holding the directional element to the footer element, such that the central axis of the lumen of the directional element is directed toward a target tissue, for example. In other embodiments, the lumen of the directional element is configured to receive one or more medical devices. The one or more medical devices may be selected from a group consisting of a cannula, a trocar, an ablation device, and an aspiration system. The directional element may also include a control knob, wherein operation of the control knob results in fixedly holding at least one of the one or more medical devices within the lumen of the directional element.

In other embodiments, the footer element may further include an interface element, the interface element located on the distal end of the footer element, and configured to interface the footer element to the tissue surface. The interface element may include a tacky surface for adherence to the tissue surface, for example.

In another aspect, a coring device includes a tubular member and a coring member. The tubular member includes a distal portion and a lumen, the distal portion of the tubular member including a finger member formed in a sidewall of the tubular member. The finger member includes a deflected configuration, such that a distal tip of the finger member is within the lumen of the tubular member, and a non-deflected configuration, such that the distal tip of the finger member is consistent with the remaining sidewall of the tubular member. The coring member may be slidably disposed within the lumen of the tubular member, and include a lumen. The coring member may be configured to advance distal to the finger member such that the finger member takes on the non-deflected configuration. The finger may take on a deflected configuration in response to a distal tip of the coring member moving proximal to the finger member. In certain embodiments, the finger member is a first finger member, the coring device further including a second finger member. Similar to the first finger member, the second finger member may be formed in the sidewall of the tubular member, the second finger member having a deflected configuration, such that a distal tip of the second finger member is within the lumen of the tubular member, and a non-deflected configuration, such that the distal tip of the second finger member is consistent with the remaining sidewall of the tubular member. The distal tip of the first finger member and the distal tip of the second finger member may contact when each of the first and second finger members are in the deflected configuration.

In other embodiments, the finger member is biased to have a deflected configuration. In still other embodiments, the coring device may further include a cylindrical member slidably positioned within the lumen of the coring member. The tubular member and the coring member may be configured to simultaneously move with respect to the cylindrical member. The coring device may further include a flat spring coupled to the tubular member and the coring member, the flat spring configured to release stored mechanical energy to simultaneously move the tubular member and the coring member, for example. The movement of the coring member with respect to the cylindrical member may create a partial vacuum within the lumen of the coring member to encourage capture of tissue therein.

In other embodiments, the tubular member and the coring member may be configured to simultaneously move in a first direction during a first time period, while the coring member is further configured to move in a second direction during a second time period, the second time period being after the first time period, and the second direction being opposite to the first direction. The first direction, for example, may be toward a target tissue.

In still other embodiments, the tubular member of the coring device is rigid. In other embodiments, the distal portion of the tubular member is rigid, and the tubular member includes a flexible portion proximal to the distal portion, such that the coring device may be advanced through a tubular structure, including a duct, of an organ of a body, such as a lung, portions of the vascular system, or portions of the digestive system.

In still another aspect, a method of obtaining a tissue sample of a target tissue includes creating a pathway to a target tissue, positioning a coring assembly adjacent the target tissue, obtaining a sample of the target tissue, and sealing the pathway. In certain embodiments, creating a pathway includes positioning a stabilization device upon a tissue surface. A medical device, such as a trocar, can be advanced along the pathway toward the target tissue. The coring assembly includes the coring device. The target tissue sample can be collected through operation of the coring device. After obtaining the desired tissue sample, sealing of the pathway may include ablating tissue adjacent to the pathway. In other embodiments, sealing the pathway includes advancing an ablation device into the pathway, and retracting the ablation device while ablating the tissue adjacent to the pathway. The ablation device may use any suitable ablation energy including, but not limited to cryogenic, radiofrequency, microwave, optical, or sonic, to name a few. In certain other embodiments, the ablation device is a bipolar RF ablation device including an elongated member having a distal portion, a proximal portion and a middle portion between the distal and proximal portions. The distal portion may include a first electrode and the middle portion may include a second electrode, the distal portion being electrically isolated from the middle portion. In other embodiments, the ablation device is a bipolar RF ablation device including an elongated member having a distal portion. The distal portion of the ablation device may include a first electrode and the coring assembly may include a second electrode. In still other embodiments, the method may further including treating the pathway with a therapeutic agent.

It is to be understood that both the foregoing general description and the following detailed description are exemplary and explanatory only and are not restrictive of the any embodiments, as claimed. Other objects, features and advantages of the embodiments disclosed or contemplated herein will be apparent from the drawings, and from the detailed description that follows below.

### BRIEF DESCRIPTION OF THE DRAWINGS

Reference will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments. In the drawings wherein like reference symbols refer to like parts:

FIG. 1 is a perspective side view of an exemplary medical system, consistent with various aspects of the present disclosure;

FIG. 2 is a perspective side view of another exemplary medical system, consistent with various aspects of the present disclosure;

FIG. 3 is a cross-section view of an element of an exemplary medical system, in accordance with certain aspects of the present disclosure;

FIG. 4 is a perspective side view of yet another exemplary medical system, consistent with various aspects of the present disclosure;

FIG. 5 is a perspective view of another element of an exemplary medical system, in accordance with certain aspects of the present disclosure;

FIGS. 6A-6C are perspective views depicting certain features of a medical device, consistent with various aspects of the present disclosure;

FIG. 7 is a side view depicting certain features of a medical device, consistent with various aspects of the present disclosure;

FIGS. 8A-8D depict side views of medical devices, in accordance with certain aspects of the present disclosure;

FIG. 9 is a perspective view of yet another exemplary medical system, consistent with various aspects of the present disclosure;

FIG. 10 is a cross-section view of certain elements of the exemplary medical system of FIG. 9, consistent with various aspects of the present disclosure;

FIG. 11 is a cross-section view of certain elements of the exemplary medical system of FIG. 9, consistent with various aspects of the present disclosure;

FIG. 12 is a flowchart depicting a method, in accordance with various aspects of the disclosure;

FIG. 13 is a perspective view of another exemplary medical system;

FIG. 14A is a view of a medical procedure performed on one lobe of a lung; and

FIGS. 14B and 14C are detailed views of a portion of the view of FIG. 14A.

### DETAILED DESCRIPTION OF THE INVENTION

Medical devices and systems, and methods of their use, are disclosed having configurations suitable for obtaining biological tissue samples suitable for analysis, such as biopsy, while minimizing undesirable collateral damage to surrounding tissue. Certain disclosed medical systems provide for obtaining biological tissue samples, while preserving organ functionality. For illustration purposes only, the disclosed medical devices and systems may be employed to consistently obtain a suitable amount of lung tissue for biopsy, the devices having elements which minimize bleeding and undesirable air leaks.

The following description is set forth for the purpose of explanation in order to provide an understanding of the various embodiments of the present disclosure. However, it is apparent that one skilled in the art will recognize that embodiments of the present disclosure may be incorporated into a number of different systems and devices.

The embodiments of the present disclosure may include certain aspects each of which may be present in one or more medical devices or systems thereof. Structures and devices shown below in cross-section or in block diagram are not necessarily to scale and are illustrative of exemplary embodiments. Furthermore, the illustrated exemplary embodiments disclosed or contemplated herein may include more or less structures than depicted and are not intended to be limited to the specific depicted structures. While various portions of the present disclosure are described relative to specific structures with respect to a medical device or system using specific labels, such as “trocar” or “cannula”, these labels are not meant to be limiting.

Reference will now be made in detail to the present exemplary embodiments, which are illustrated in the accompanying drawings.

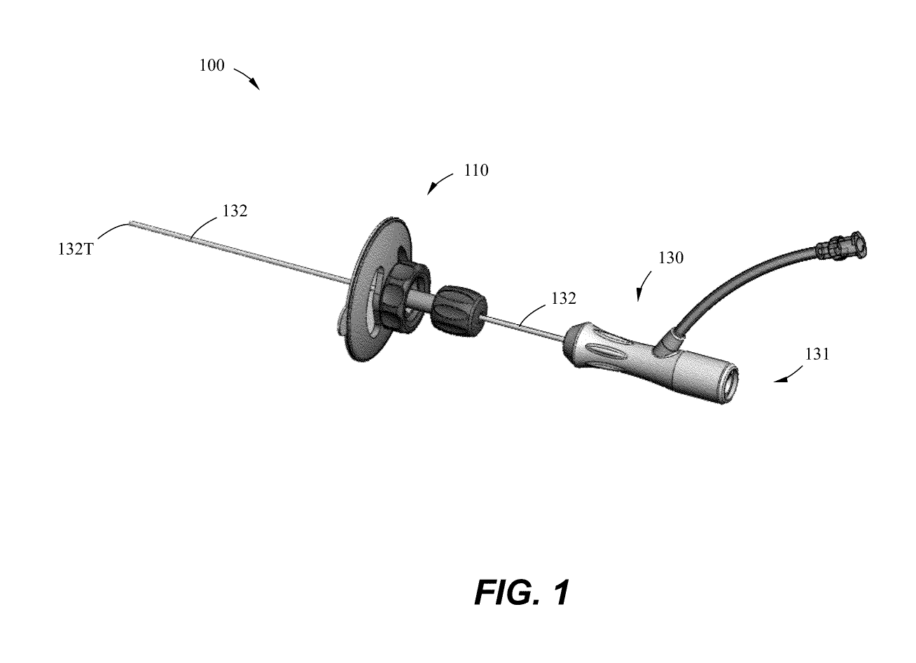

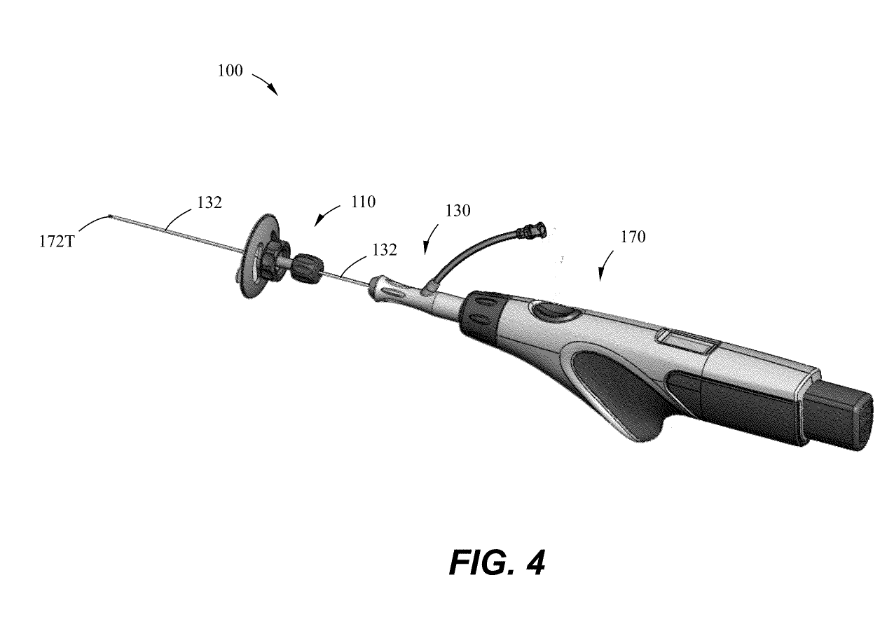

Turing to FIG. 1, a medical system 100 generally includes a stabilization device 110 and a cannula device 130. The stabilization device 110 is configured to engage a tissue surface, skin for example, and provide a stable platform from which other devices or tools can be advanced to approach and engage a target tissue. Stabilizer device 110 may be constructed from materials compatible with one or more Viewing Technologies, as described above. The cannula device 130 may include a proximal portion configured to interface with one or more medical devices, as described in greater detail below with respect to FIG. 4, for example. The cannula device 130 includes a cannula 132 extending therefrom, the cannula 132 including a lumen for the transport of therapeutic agents or medical devices therethrough. While cannula 132 is described in terms of having a single lumen, cannula 132 may include multiple lumens, each lumen configured for one or more different tasks. Such tasks, for example, may include advancement of medical devices toward a target tissue within a body, aspiration of tissue or fluids, deposition of therapeutic agents such as drugs to address the target tissue, or similar tasks. The cannula 132 of the cannula device 130 extends through the stabilization device 110 and ends in a tip 132T. The cannula 132 may be constructed from any suitable material and of any suitable size to provide the necessary access way to a target tissue, keeping in mind a therapeutic device that may be employed in the future procedure. The cannula device 130 may be constructed from materials that are compatible with one or more Viewing Technologies, as described above, to allow for more accurate positioning of the cannula 132, for example. For illustration purposes only, the one or more lumens of cannula 132 may each have a working or inner diameter from about 0.006 inches to about 0.135 inches.

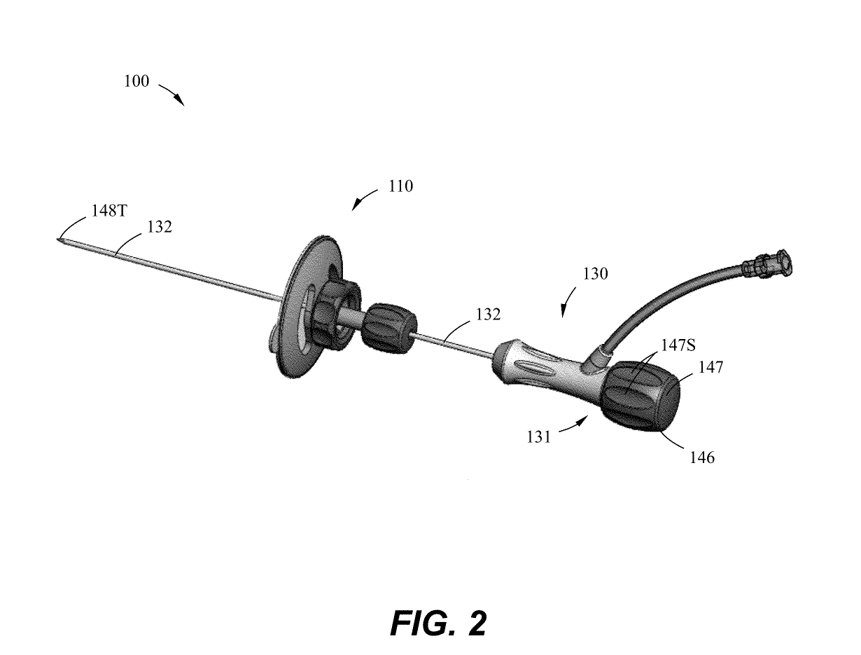

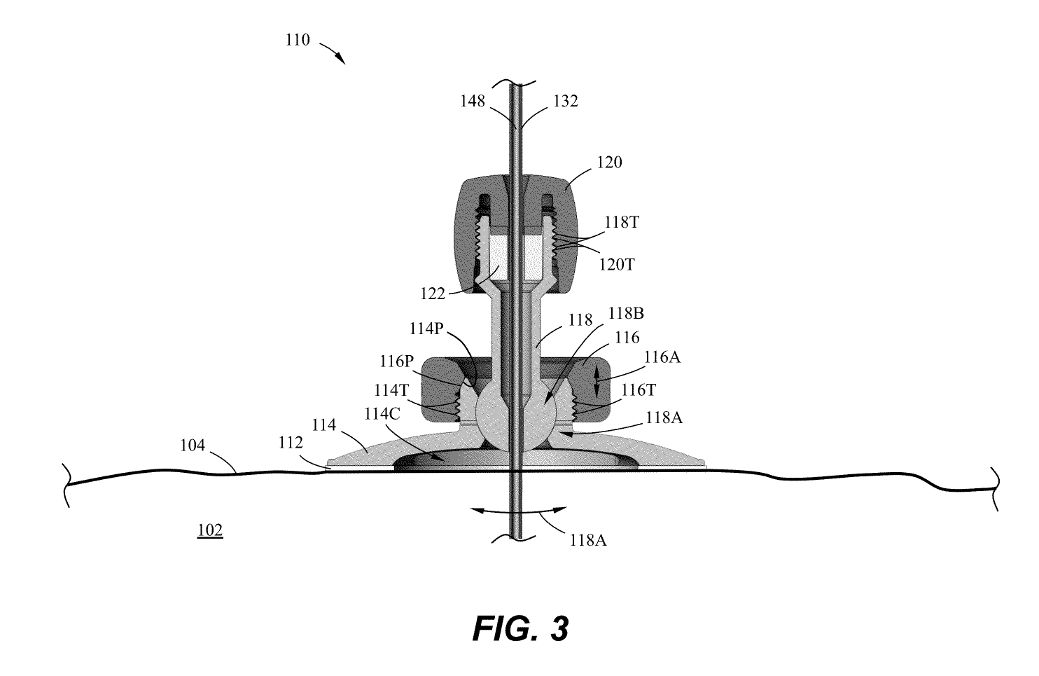

Now turning to FIG. 2, the system 100 may further include a trocar 146. The trocar 146 may include a handle or proximal portion 147 that interfaces with the proximal end 131 of cannula device 130, and a shaft 148, passing through a lumen of cannula 132, and ending in a sharp distal tip 148T. The handle portion 147 may include various surface features 147S to enable better control or manipulation of the trocar 146 during use. The tip 148T of the shaft 148 of the trocar 146 is sized to translate within the lumen, e.g. at least one lumen, of the cannula 132, and may be employed to create access ports in the thoracic area, or other area, of a body. As is described in greater detail below with respect to FIG. 3, once the stabilization device 110 is positioned upon a tissue surface, the cannula device 130/trocar 146 combination may be advanced to create a therapeutic port or pathway between the tissue surface and a location within the thoracic area or other bodily structure, adjacent to or within a target tissue for example. As with the cannula device 130, the trocar 146 may be constructed of materials that are compatible one or more Viewing Technologies described above.

Now turning to FIG. 3, stabilization device 110 may include an interface element 112 configured to interface between the stabilization device 110 and a tissue surface 104 of biological tissue 102. It should be noted that the biological tissue 102 may include skin, or may include other tissues of the body. For example, if the device 100 is utilized during another procedure where the body cavity is opened to expose other tissue, biological tissue 102 may include such tissue. In this case, for illustration purposes, biological tissue 102 may include muscle tissue, or tissues of one or more internal organs such as the stomach, lung, or liver, for example. The interface element 112 may include features to allow the stabilization device 110 to be attached to, and maintain its position upon, the tissue surface 104. For illustration purposes only, the interface element 112 may include a tacky surface to momentarily adhere the stabilization device 110 to the tissue surface 104 at a desired location. The interface element 112 may be integrated into the stabilization device, or may be a separate device applied to the tissue surface 102 prior to positioning the stabilization device 110.

The stabilization device 110 may further include a footer element 114 and associated control knob 116, and directional element 118 and associated control know 120. As depicted, a proximal portion of the footer element 114 includes teeth portions 114T, which engage teeth portions 116T of the control knob 116. Rotating the control knob 116 moves the control knob 116 in a longitudinal direction relative to the proximal portion of the footer element 114, as generally depicted by arrow 116A. The proximal portion of the footer element 114 is configured as a socket 114S to receive a distal bulbous end 118B of the directional element 118. As the control knob 116 translates distally, a surface 116P of the control knob 116 engages a surface 114P of the footer element 114 forcing the socket 114S to engage the distal end 118B of the directional element 118, locking the direction element 118 in place, upon sufficient force. In this way, once the stabilization device 110 is positioned upon a tissue surface, the directional element 118 can be configured to define an approach vector toward a target tissue. The ball (118B) and socket (114S) joint formed by the directional element 118 and the footer element 114 allows for three-dimensional positioning of a longitudinal axis of the directional element 118, and the cannula 132 and medical device 148 passing therethrough for example, as suggested by arrow 118A. Such a stabilization system 110 assists to maintain a stable access point during required physical motion of the patient, such as respiratory functions for example.

A proximal portion of the directional element 118 interfaces to the control knob 120 through teeth 118T and 120T, respectively. The control knob 120 may include a compressible material 122 configured to engage the cannula 132, or other device provided therethrough. Accordingly, as the control knob 120 translates distally with respect to the directional element 118, the compressible material compresses about the cannula 132, holding the cannula 132 stationary with respect to the stabilization device 110, the tip 132T of the cannula 132 being held adjacent the target tissue site for example.

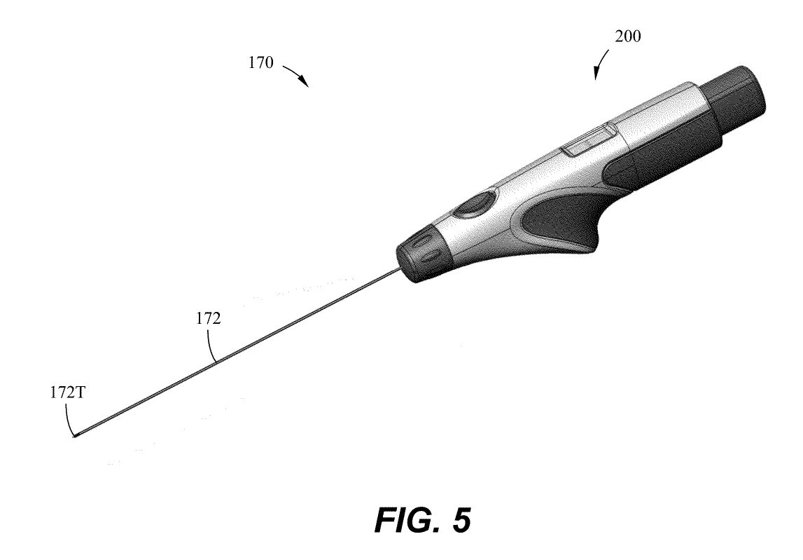

Now turning to FIG. 4, once an access pathway to the target tissue is created, the trocar 146 may be removed and another therapeutic device may be interfaced to system 100. As depicted in FIG. 4, the system 100 further includes a tissue-extracting device 170. The tissue-extracting device 170 may include a tubular member 172 extending through a lumen of the cannula 132 of the cannula device 130 and ending in a tip 172T, which extends out the distal opening of the cannula 132. The tubular member 172 is preferably stainless steel, but may be constructed from any suitable material, such as one or more materials compatible with Viewing Technologies, as described above. Turning also to FIG. 5, the tissue-extracting device 170 includes a handle portion 200 from which the tubular member 172 extends and terminates in a tip 172T. The tip 172T is positioned adjacent or within a target tissue from which tissue is to be extracted for purposes of analysis, a biopsy for example.

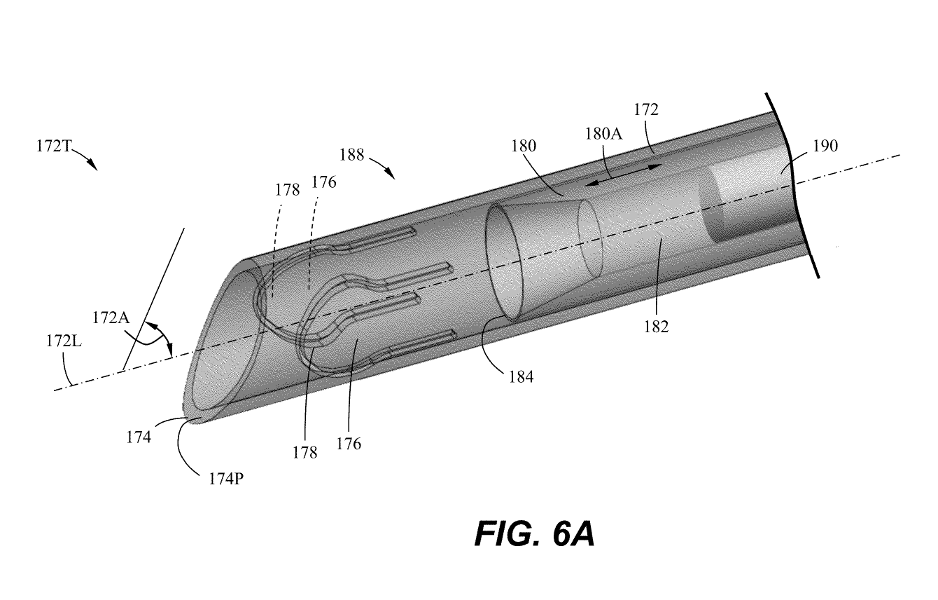

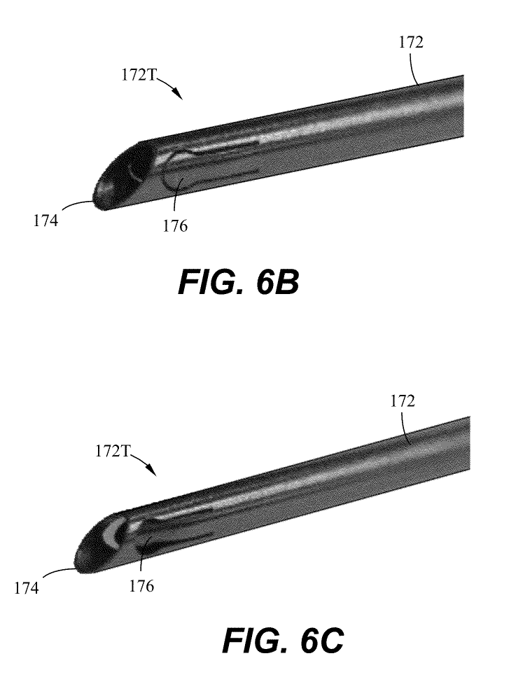

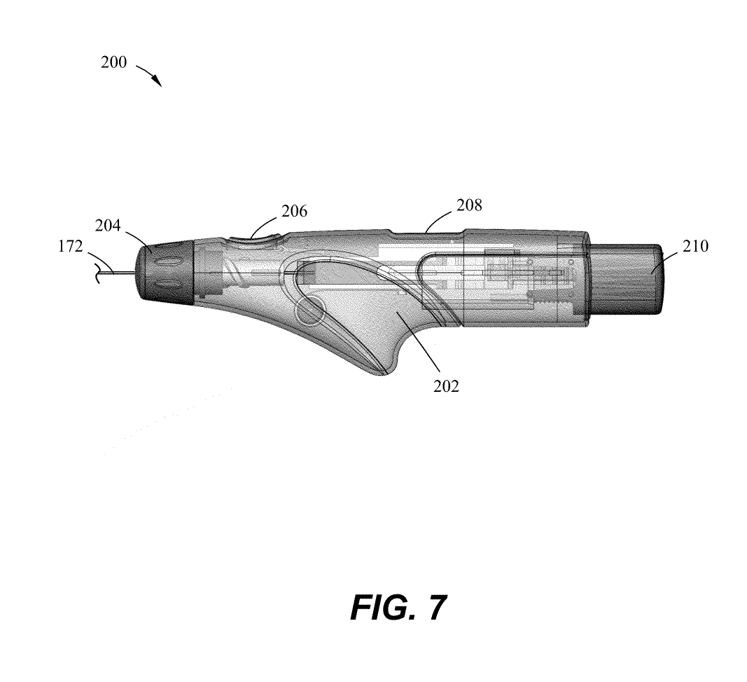

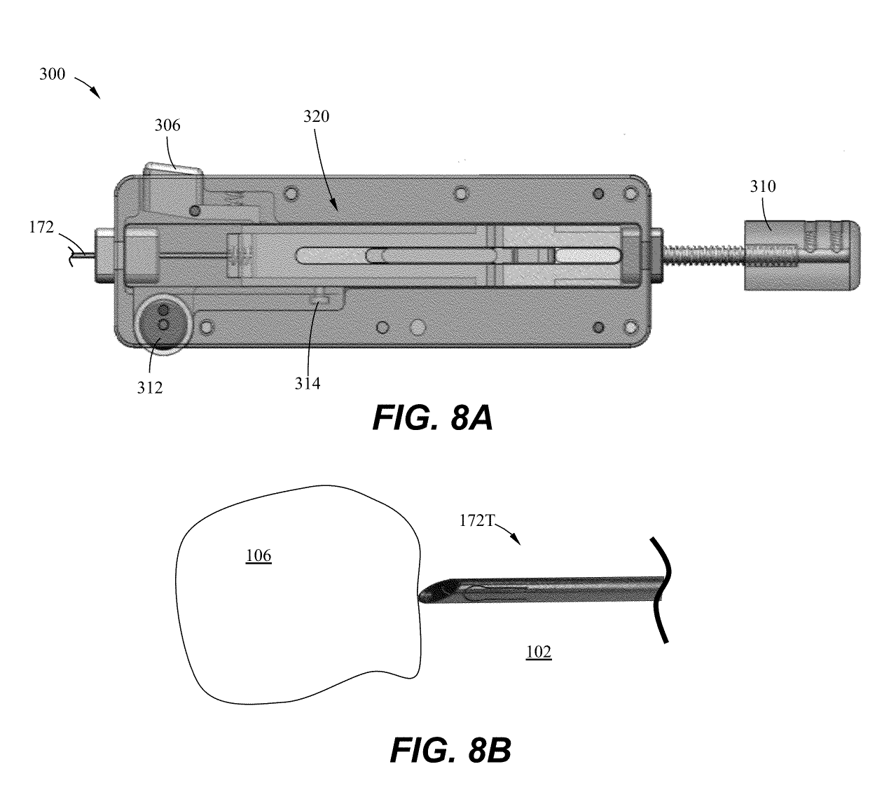

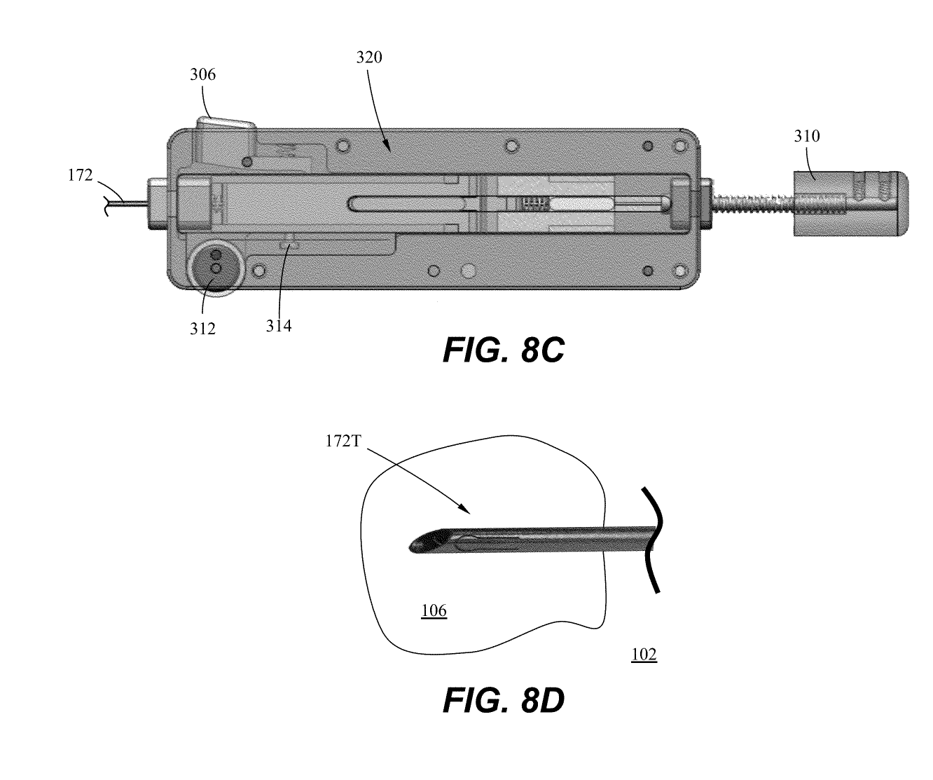

Turning now to FIG. 6A, tip 172T of the tubular member 172 of the tissue-extraction device 170 is depicted in translucent form for purposes of discussion only. Tip 172T includes a distal end 174 terminating in a sharpened portion configured to engage a desired target tissue. A planar surface 174P at the distal tip 174 may define an angle 172A with respect to a central longitudinal axis 172L of the tubular member 172, the angle 172A determined to enhance penetration of the target tissue, for example. Accordingly, the angle 172A may be a first value for a first type of target tissue and a second value for a second type of target tissue. The angle 172A may, for illustration purposes only, be from about 90° to 20°. The tip portion 172T may also include finger members 176 employed to retain a portion of a target tissue to be extracted, as described in greater detail below with respect to FIGS. 7 and 8. Each of the finger members 176 may be configured to have a deflected configuration at rest such that the tip portions 178 of each member 176 are distanced from each other less than a diameter of the tubular member 172 immediately distal to members 176. The members 176 may be formed through any suitable manner, for example through laser cutting of the tubular member 172. The tip portions may have geometric dimensions different that the remainder of member 176. For example, the tip portion 178 may be form a circular end of member 176 having a diameter greater than a width of the remainder of member 176, as generally shown in FIG. 6A. To ensure proper movement of the finger members 176, the laser may be positioned such that the laser travels in the direction of the desired deflection of the finger members 176 during laser cutting. The deflectable configuration of the members 176 may be imparted through heat-treating the stainless steel of members 176, for example. The geometric shapes of the tip portions 178 are selected to allow the tip portions 178 to contact each other, when in a deflected configuration, over a sufficient surface area such that the obtained tissue will remain within the tissue-extracting device 170. Accordingly, the tip portions 178 preferably include curved portions when viewed laterally toward and perpendicular to the finger member 176 surface.

The tissue-extracting device 170 further includes a tubular coring member 180 slidably disposed within the lumen of tubular member 172, as generally indicated by arrow 180A. The tubular member 172 and the coring member 180 may also be referred to as the coring assembly 188. The coring member 180 may include a sharpened distal portion 184, which may be employed to encourage target tissue to enter the lumen 182 of the coring member 180 during tissue extraction. A solid cylindrical member 190 is slidably disposed within the lumen of the coring member 180. Cylindrical member 190 has an outer diameter approximately equal to the inner diameter of the coring member 180, as depicted. The movement of tubular member 172, coring member 180, and cylindrical member 190 is provided in a coordinated fashion to obtain a tissue sample of the target tissue having a consistent tissue volume suitable for further analysis, as is discussed in greater detail below with respect to FIGS. 8A-8D.

For clarity, the finger members 176 are depicted in a non-deflected position. However, as mentioned above, the normal resting position for members 176 is a deflected position, as best shown in FIG. 6C. As should be readily understood, while the members 176 are normally in a deflected position, as the coring member 180 translates within the lumen of the tubular member 172 to a point where the tip 184 of the coring member 180 is distal to the members 176, the members 176 are held in a non-deflected configuration by the outer wall of the coring member 180. Accordingly, as the coring member 180 is retracted proximal to the finger members 176, the members 176 are then allowed to take on their deflected configuration, as generally depicted in FIG. 6C.

Turning also to FIG. 6B, the tip 172T of tubular member 172 is depicted with members 176 in a non-deflected configuration, the coring member 180 slidably disposed adjacent to the members 176 for example. Now turning also to FIG. 6C, once the coring member 180 is refracted, the members 176 are then allowed to assume their retracted configuration. Accordingly, the coring member 180 may be positioned within the lumen of the tubular member 172 such that the members 176 are in a non-deflected configuration.

In operation, the coring member 180 is slidably disposed such that the distal end of the coring member 180 is distal to the tips 178 of the fingers 176, the coring member 180 holding the members 176 in a non-deflected position, as best depicted in FIG. 6B. In unison, the tubular member 127 and the coring member 180, i.e. the coring assembly 188, are simultaneously advanced within the target tissue. The coring assembly 188 is advanced into the target tissue, the movement of the coring assembly 188 preferably measured with respect to the handle 200, otherwise referred to as a coring action, while the cylindrical member 190 is stationary with respect to the handle 200, the coring assembly 180 advancing over the member 190. Advancing the coring assembly 188 while maintaining the cylindrical member 190 substantially stationary may create a vacuum in the lumen 182 of the coring member 180 helping to encourage the capture and detainment of at least a portion of the target tissue therein. The depth the coring assembly 188 enters the target tissue may be controlled by providing a hard stop with respect to such mechanical movement. In this way, the total travel limit of the coring assembly 188 may be limited to, for illustration purposes only, from about 5 mm to about 30 mm. After the coring action, the coring member 180 can then be slidably moved in a proximal direction within the lumen of the tubular member 172 until the distal tip 184 passes proximal to the distal end 178 of the members 176, allowing the members 176 to take on their deflected configuration, as best shown in FIG. 6C, and acting to hold, or otherwise retain, the tissue within the tissue-extracting device 170. The initial movement of the coring assembly 188 within the target tissue, followed by the immediate retraction of the coring member 180 with respect to the tubular member 172 is preferably automated to ensure that the characteristics of the tissue sample collected, as well as the repeatability of such tissue collection, is consistent for each tissue sample collected. Once the distal end 172T of the tissue-extracting device 170 is out of the patient's body, the cylindrical member 190 may be distally moved within the lumen 182 of the coring element 180 to expel the extracted target tissue from the tissue-extracting device 170.

Now turning to FIG. 7, the handle 200 is depicted in greater detail. Handle 200 may include a grip portion 202 employed to better control the tissue-extracting device 170 during use. Handle 200 may also include a number of controls 204, 206, 208 and 210 to facilitate various features of the tissue-extracting device 170. For illustration purposes only, a control 204 may cooperate with other gearing to adjust the hard stop and ultimately the length of travel the coring assembly 188 slidable moves during the coring action, to define a desired volume of a portion of target tissue to be extracted, as discussed above. Another control 206 may be employed to facilitate controlled movement of the coring assembly 188 in a cooperative manner, for example to obtain the tissue sample. A control 208 may be employed to store energy for the coring action, as is discussed in greater detail below with respect to FIGS. 8A-8D. For example, the control 208 may cooperate with a spring member to store potential energy suitable for providing the coring action. Preferably, a flat spring is utilized since such a spring provides for a more consistent force over its motion as it releases stored energy. Last, a control 210 may be employed to advance the cylindrical member 190 with respect to the coring assembly 188 and expel the target tissue sample from the tissue-extracting device 170.

Now turning to FIG. 8A, another handle 300 will be described. Handle 300 is similar to handle 200, with controls 306, 308, and 310. Control 308, as part of a coring actuator 320, is slidably moved in a proximal direction to store energy in flat spring 312 attached thereto through attachment point 314, a fastener for example. Now also referring to FIG. 8B, the distal tip 172T of the tubular member 172 of the tissue-extracting device 170 is positioned within tissue 102 and adjacent a target tissue 106. The deflectable members 176 of the tubular member 172 are in a non-deflected position, the tip 184 of the coring member 180 (not shown) being distal to the distal end of the tubular members 176. The switch 306 mechanically holds the coring actuator 320 in this activated mode, the proximal end of the switch 306 engaging a detent of the coring actuator 320, for example, as depicted.

Now turning to FIG. 8C, upon activation of the switch 306, the energy stored in flat spring 312 is released and the coring actuator 320 instantaneously translates distally to cause the coring assembly 188 to enter the target tissue, obtaining a portion thereof. The control 310 may then be employed to retract the coring member 180 proximally with respect to the finger members 176. The members 176 are then free to take on their deflectable configuration to retain the sample of target tissue obtained. The tissue-extraction device 170 can then be removed from the patient's body, and the control 310 can then be activated to advance the cylindrical member 190 distally out the coring assembly 188, expelling the tissue sample therefrom. Since the flat spring provides a constant force during motion, e.g. release of energy stored therein, a tissue sample of consistent volume can be obtained in a repeatable manner.

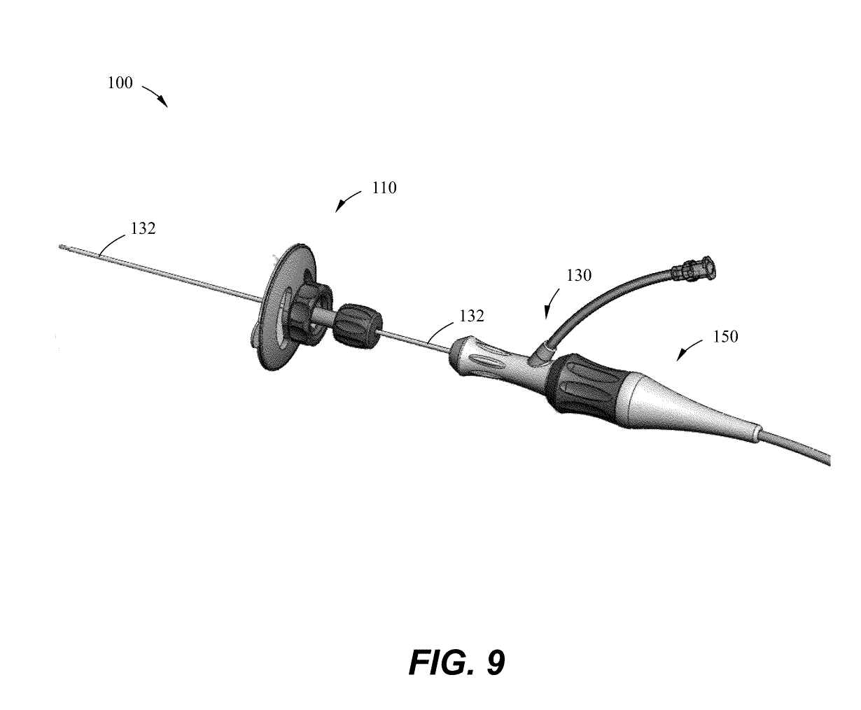

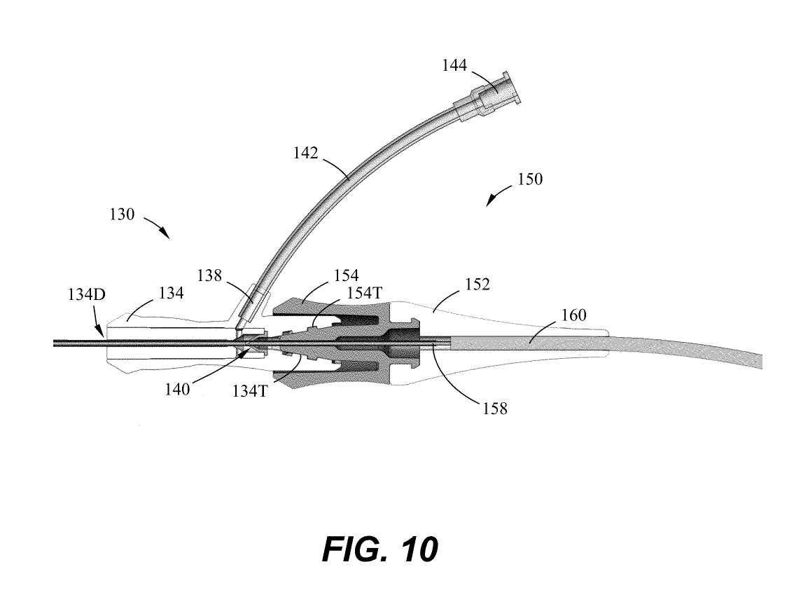

Now turning to FIG. 9, the tissue-extracting device 170 has been removed from the medical system 100, the medical system 100 now including a therapeutic device 150. Therapeutic device 150, for example, may be a radiofrequency (RF) ablation device 150 employed to treat the target tissue surrounding the area from which the tissue sample was retrieved. Such tissue treatment, for example, may minimize undesirable bleeding and/or minimize air leaks, e.g. where the target tissue is lung tissue. Now turning to FIG. 10, the cannula device 130 and the RF ablation device 150 are depicted in cross-section. The cannula device 130 includes a handle portion 134 through which the cannula 132 passes, the handle portion 134 ending in a distal portion 134D. The handle portion 134 includes an interface 138 configured to accept a tube 142 ending in a port 144. The port 144 is in fluid communication with the lumen of the cannula 132 such that fluids provided to port 144 are also provided to the cannula 132. Such fluids may be used to help facilitate RF ablation of target tissue surrounding the tissue sample site, or may be used to transport therapeutic agents to the target tissue site. Alternatively, such fluids may include biological tissue adhesives to provide a sealing action. Such compatible adhesives may be used alone or in combination with the RF ablation tool to treat the target tissue sample site. The cannula device 130 may include a one-way value 140 to prevent such fluids from travels out the proximal end of the cannula device 130. A handle portion 154 of the therapeutic device 150 may be attached to the cannula device 130 through a snap-fit connection or a threaded connection, utilizing teeth 134T and corresponding teeth 154T for example. The therapeutic device 150 may also include a member 152 which may be used to interface cable 160 to the handle portion 154 and provide strain relief to the cable 160. Conductors 158 carried as part of the cable 160 may be advanced through the cannula 132 of the cannula device 130, the distal end of the conductors 158 forming an RF probe.

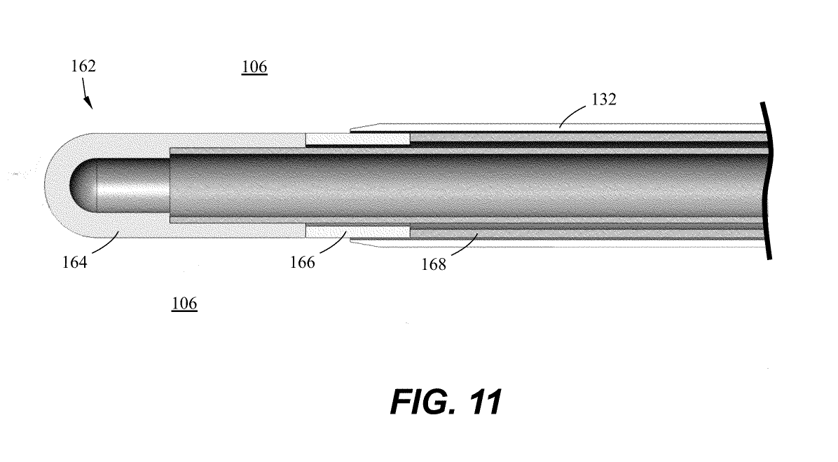

Now turning to FIG. 11, an exemplary RF probe 162 is a bi-polar probe including a first conductor 164, an insulator 166 and a second conductor 168. In operation and with the aide of the stabilization device 100, the RF probe 162 is positioned within the target tissue 106 at the location the tissue sample was obtained to treat the area to prevent undesirable bleeding or air leaks, or provide other therapeutic treatments at the target tissue site. The RF probe 162 is extended out the distal end of the cannula 132 such that the cannula does not make electrical contact with the first conductor 164. The first conductor 164 can then be energized and the second conductor 168 may be grounded to provide a current there between to ablate adjacent target tissue. The second conductor 168 may be electrically coupled to the cannula 132 such that ablative current flows from the first conductor 164 to the cannula 132, as well. The second conductor 168, and the cannula 132, may be both grounded for example.

The RF probe 162 and the cannula 132 are then retracted as a unit as the target tissue 106 is ablated. The medical system 100 may include a feedback system to ensure the tissue is properly ablated as the RF probe 162 is refracted. For example, the RF probe 162 may include a temperature sensor (not shown) configured to measure the temperature of the first conductor 164, for example, providing an indicating when the adjacent target tissue 106 is sufficiently ablated. Such a feedback system may provide a visual or audible indicator to assist a surgeon in maintaining proper speed of the RF probe as it is retracted from the target tissue 106. While depicted as a bipolar probe, one of ordinary skill will appreciate that a monopole probe may be employed, a grounding pad provided on the patients back for example.

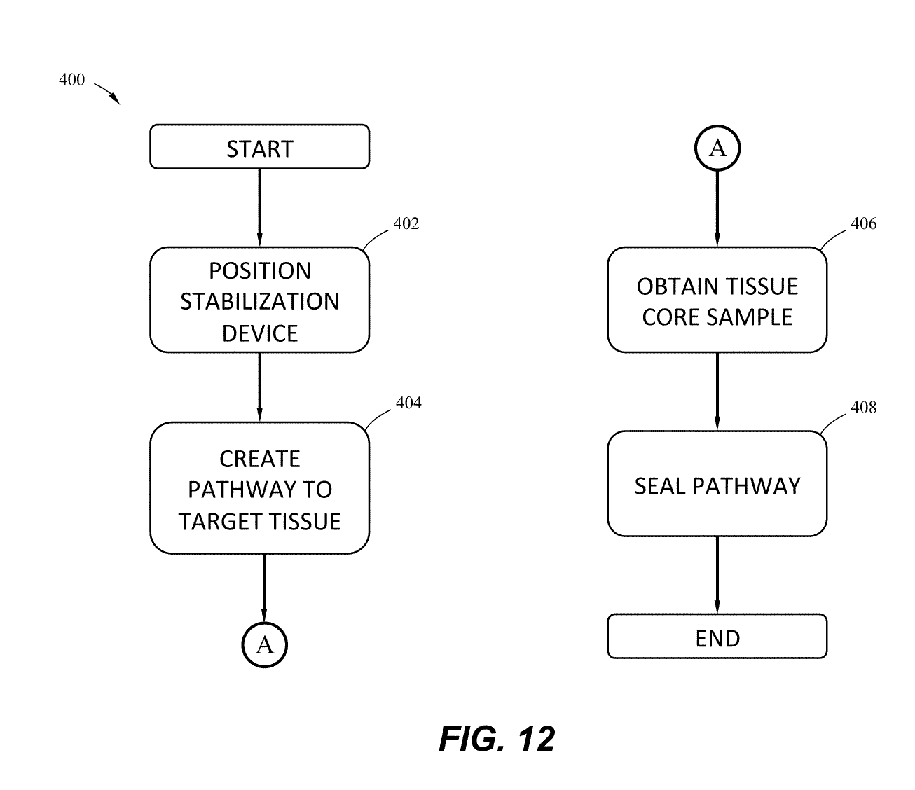

Now turning to FIG. 12, a method 400 of obtaining a tissue sample will be described. In a first step 402, a stabilization device, such as stabilization device 110, is provided to provide access to a desired target tissue site. A pathway is then created in a step 404, through the use of a trocar for example. A target tissue sample is then obtained in a step 406, employing devices described herein for example. The target tissue immediately adjacent the obtained tissue sample is then treated, to prevent undesirable bleeding for example, in a step 408, employing devices, systems and methods described herein.

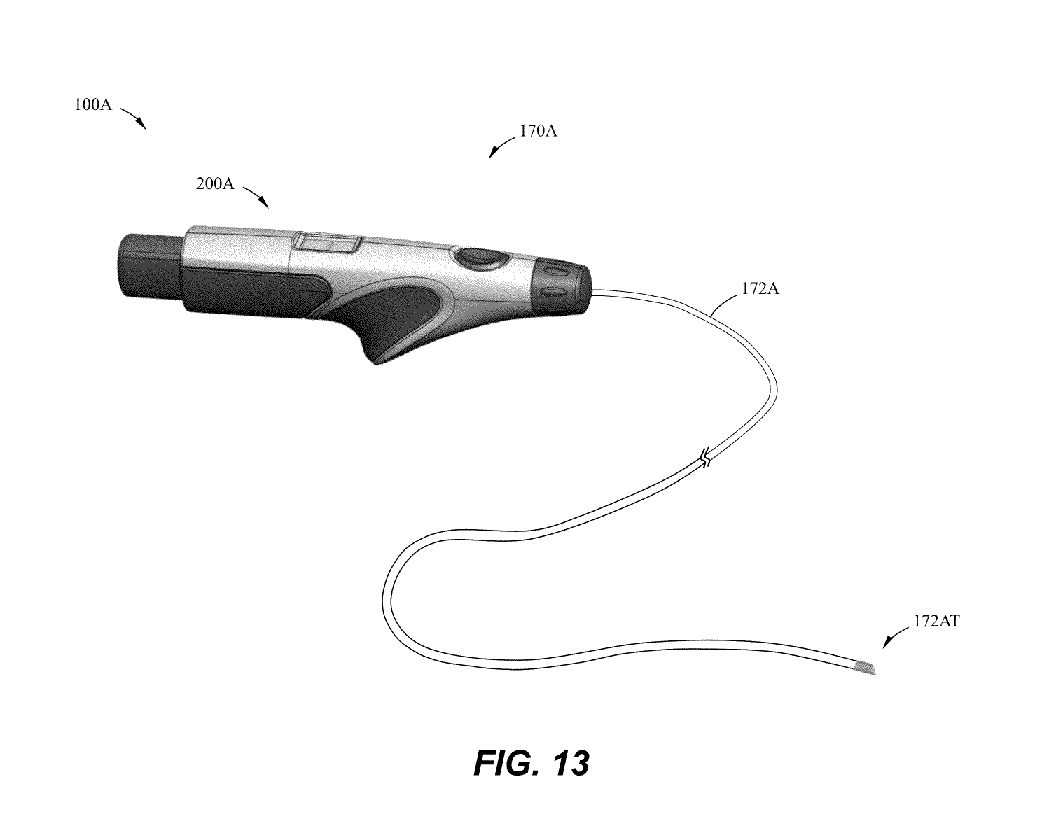

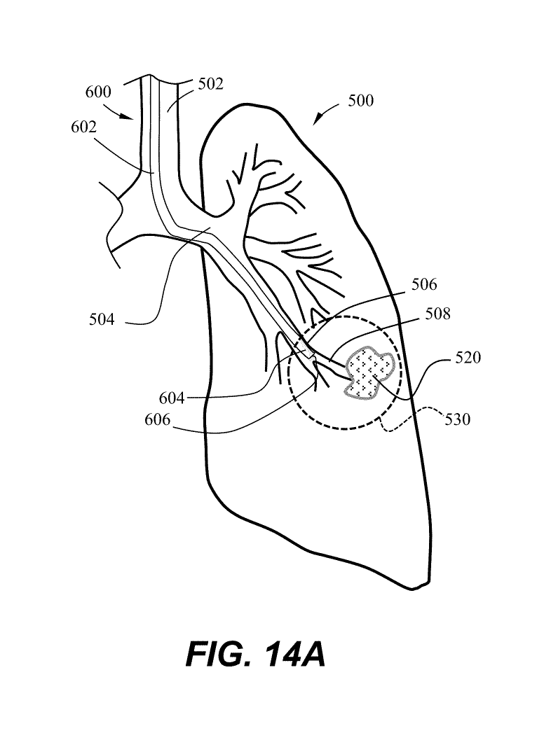

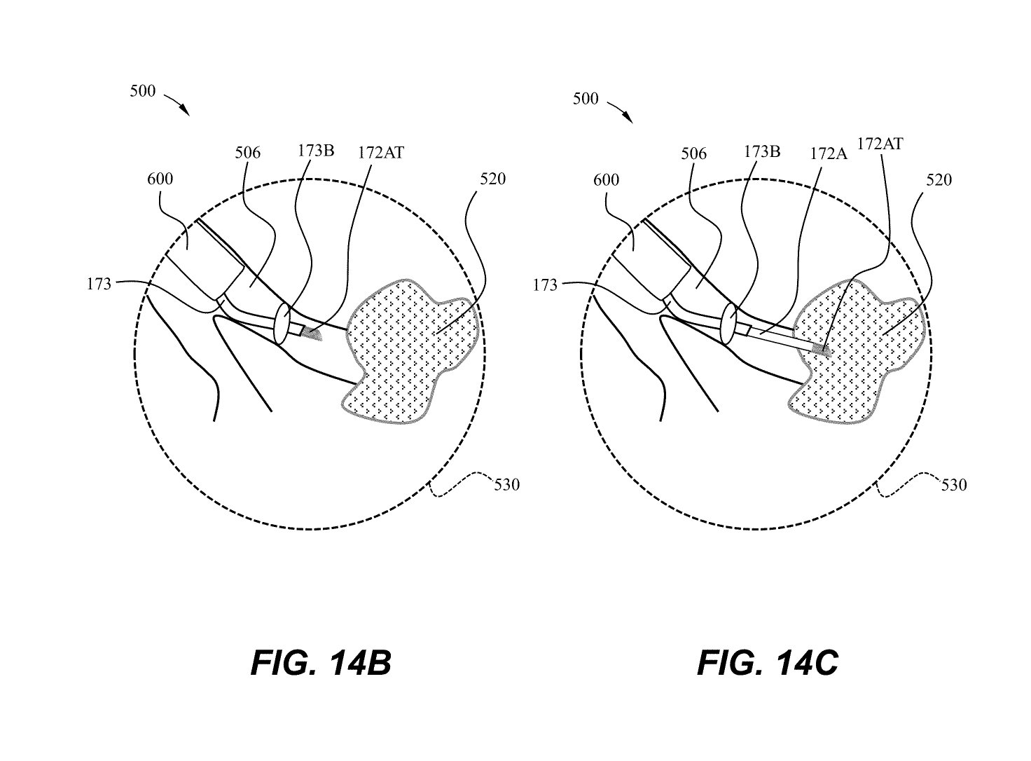

Turning now to FIG. 13, another medical system 100A, is similar to medical system 100, however includes tissue-extracting device 170A. Tissue-extracting device 170A is similar to tissue-extracting device 170, but rather than a tubular member 172, which may be rigid for example, the tissue-extracting device 170A may include a tubular member 172A that is flexible, and may have a longer length, adapting the tubular member 172A to be able to navigate through bodily structures, such as the airways of the lung for example. The tubular member 172A may be formed from any suitable biocompatible material that imparts a desired flexibility to allow access through navigation systems, as described below with respect to FIGS. 14A-C. For example, the tubular member 172A may be formed from flexible glass, metal, or plastics, such as Peek polymers offered by Victrex for example. The tubular member 172A may end in a tip portion 172AT, which may be similar in construction and operation to tip 172 of tissue-extracting device 170, for example, including members 172 or such similar structures, to cooperate with other tubular structures, similar to coring member 180 of tissue-extracting system 170 for example, to allow for the collection and retaining of biological tissue for analysis, e.g. a biopsy, as discussed with respect to medical system 100. The tip 172AT, however, which may include a rigid or semi-rigid body, may have a length adapted to define a minimum deflection radius of the tubular member 172A. Accordingly, the tip 172AT of tissue-extracting device 170 may have a length that is shorter than the tip 172T of tissue-extracting device 170. The tubular member 172A and tip 172AT may have outer diameters approaching a few millimeters, for example from between 2 and 5 millimeters, allowing the tip 172AT to reach certain target tissues that otherwise could not be as readily reached, due to size constraints of the medical devices utilized for example.

With reference to FIG. 14A, a medical procedure to obtain a lung 500 tissue biopsy of a portion of a target tissue 520 within a tissue region 530 will be described. General access to the target tissue 520 may be provided by a steerable navigation system 600, such as the Electromagnetic Navigation Bronchoscopy® i•Logic® System manufactured by superDimension, Ltd. While such systems may provide enhanced ability to access locations within a body, access may be limited by the size of the tubular structures that make up the navigation system. The steerable navigation system 600 may include a tubular structure 602 having a distal end 604 and distal opening 606. The navigation system 600 may be advanced through a trachea 502 and a left stem bronchus 504 of the lung 500. The distal end 604 may be further advanced or manipulated into a bronchi or bronchioles 506 of the lunch 500, providing the distal opening 604 at a location near the target tissue 520, the distal end 604 of the tubular structure 602 not permitted to pass into a bronchi or bronchioles 508 for example, as generally shown.

With reference also to FIG. 14B, which depicts detail of tissue region 530, the medical system 100A may include a tubular member 173 that provides more specific access to the target tissue 520 from the distal opening 606 of the tubular member 602 of navigation system 600. The tubular member 173 may include an inflatable structure 173B near its distal end to facilitate support for the tubular member 173 when inflated to engage surrounding tissue, e.g. the inner wall surfaces of the bronchi or bronchioles 508. With the tubular 173 stabilized within the bronchi or bronchioles 508, the tubular member 172A may be advanced, the tip 172AT of the tubular member 172A shown advanced out of a distal opening of the tubular member 173. With reference also to FIG. 14C, the tubular member 172A may be further advanced, in accordance with the various aspects of this disclosure, such that the tip 172AT is advanced within the target tissue 520 obtaining a tissue biopsy thereof. The medical system 100A may then be removed, as well as the tubular member 173 and navigational system 600.

It should be apparent to one of ordinary skill in the art that the tubular member 173 may be an integral part of the tissue-extracting system 170A, the tubular member 172A being slidably disposed within the tubular member 173 for example. In this case, the proximal portions of both tubular members 172A, 173 may terminate in the handle portion 200A of system 170A, the handle portion 200A including controls, for example, allowing for the independent advancement of each of the tubular members 172A, 173, as well as inflation and deflation of the balloon 173B. Alternatively, the tubular member 173 may be a medical device independent of the medical device 170A, the tubular member 173 being positioned simultaneously with or prior to advancement of the tubular member 172A for example.

If desired, consistent with the various embodiments and methods of their use disclosed or contemplated herein, an RF probe, such as RF probe 162 of FIG. 11 may be positioned within the target tissue 520 (not shown) at the location the tissue sample or biopsy was obtained to treat the area to prevent undesirable bleeding or air leaks, or provide other therapeutic treatments at the target tissue site. The tip of the RF probe 162, for example, may be extended out the distal end of the tubular member 173. A portion of the tubular member 173 may include a metallic coating to provide for a bi-polar operation of the RF probe 162, the metallic portion of the tubular member 173 being a second conductor 168A, such that the first conductor 164 can then be energized and the second conductor 168A may be grounded to provide a current there between to ablate adjacent target tissue.

As discussed above with respect to FIG. 11, the RF probe and the tubular member 173 may be retracted as a unit as the target tissue 106 is ablated. The medical system 100A may, as described above with respect to medical system 100, include a feedback system to ensure the tissue surrounding the target tissue 520 is properly ablated as the RF probe is refracted. For example, the RF probe 162 may include a temperature sensor (not shown) configured to measure the temperature of the second conductor, for example, providing an indicating when the adjacent target tissue 106 is sufficiently ablated. Such a feedback system may provide a visual or audible indicator to assist a surgeon in maintaining proper speed of the RF probe as it is retracted from the target tissue 106. While depicted as a bipolar probe, one of ordinary skill will appreciate that a monopole probe may be employed, a grounding pad provided on the patients back for example.

While the embodiments have been described in conjunction with several specific examples, it is evident to those skilled in the art that many further alternatives, modifications and variations will be apparent in light of the foregoing description. Thus, the embodiments described herein are intended to embrace all such alternatives, modifications, applications and variations as may fall within the spirit and scope of the appended claims.

What is claimed is:

1. A stabilization device, comprising:
a footer element having distal and proximal ends, the distal end configured to engage a tissue surface, the proximal end of the footer element including a socket;
a directional element having distal and proximal ends, and a lumen therethrough, the lumen of the directional element including a central axis, the distal end of the directional element being configured to be coupled with the socket of the proximal end of the footer element.

2. The device of claim 1, wherein the footer element further includes a control knob, operation of the control knob results in fixedly holding the directional element to the footer element, wherein the central axis of the lumen of the directional element is directed toward a target tissue.

3. The device of claim 1, wherein the lumen of the directional element is configured to received one or more medical devices.

4. The device of claim 3, wherein the one or more medical devices are selected from a group consisting of a cannula, a trocar, an ablation device, and an aspiration system.

5. The device of claim 3, the directional element further including a control knob, wherein operation of the control knob results in fixedly holding at least one of the one or more medical devices within the lumen of the directional element.

6. The device of claim 1, wherein the footer element further includes an interface element, the interface element located on the distal end of the footer element, the interface element configured to interface the footer element to the tissue surface.

7. The device of claim 6, wherein the interface element includes a tacky surface.

8. A coring device, comprising:
a tubular member having a distal portion and a lumen, the distal portion of the tubular member including a finger member formed in a sidewall of the tubular member, the finger member having a deflected configuration, such that a distal tip of the finger member is within the lumen of the tubular member, and a non-deflected configuration, such that the distal tip of the finger member is consistent with the remaining sidewall of the tubular member; and
a coring member slidably disposed within the lumen of the tubular member, the coring member having a lumen, the coring member configured to advance distal to the finger member such that the finger member takes on a non-deflected configuration,
wherein the finger takes on the deflected configuration in response to a distal tip of the coring member moving proximal to the finger member.

9. The device of claim 8, wherein the finger member is a first finger member, the device further including a second finger member, the second finger member being formed in the sidewall of the tubular member, the second finger member having a deflected configuration, such that a distal tip of the second finger member is within the lumen of the tubular member, and a non-deflected configuration, such that the distal tip of the second finger member is consistent with the remaining sidewall of the tubular member.

10. The device of claim 9, wherein the distal tip of the first finger member and the distal tip of the second finger member make contact when each of the first and second finger members are in the deflected configuration, respectively.

11. The device of claim 8, wherein the finger member is biased to have a deflected configuration.

12. The device of claim 8, wherein the device further includes a cylindrical member slidably positioned within the lumen of the coring member.

13. The device of claim 12, wherein the tubular member and the coring member are configured to simultaneously move with respect to the cylindrical member.

14. The device of claim 13, further comprising a flat spring coupled to the tubular member and the coring member, the flat spring configured to release stored mechanical energy to simultaneously move the tubular member and the coring member.

15. The device of claim 12, wherein movement of the coring member with respect to the cylindrical member creates a partial vacuum within the lumen of the coring member.

16. The device of claim 8, wherein the tubular member and the coring member are configured to simultaneously move in a first direction during a first time period, the coring member being further configured to move in a second direction during a second time period, the second time period being after the first time period, the second direction being opposite to the first direction.

17. The device of claim 16, wherein the first direction is toward a target tissue.

18. The device of claim 8, wherein the tubular member is rigid.

19. The device of claim 8, wherein the distal portion of the tubular member is rigid, the tubular member including a flexible portion proximal to the distal portion, such that the device may be advanced through tubular structures of an organ of a body.

20. The device of claim 19, wherein tubular structures includes bronchi of the lung, veins and arteries of the vascular system, a duct of an organ, or a tubular structure of an organ of the digestive system.

21. A method, comprising:
creating a pathway to a target tissue;
positioning a coring assembly adjacent the target tissue;
obtaining a sample of the target tissue;
sealing the pathway.

22. The method of claim 21, wherein creating a pathway includes positioning a stabilization device upon a tissue surface.

23. The method of claim 22, wherein creating a pathway includes advancing a trocar toward the target tissue.

24. The method of claim 21, wherein the coring assembly includes the coring device of claim 8.

25. The method of claim 24 wherein obtaining a sample of the target tissue includes operating the coring device.

26. The method of claim 21, wherein sealing the pathway includes ablating tissue adjacent to the pathway.

27. The method of claim 21, wherein sealing the pathway includes:
advancing an ablation device into the pathway; and
retracting the ablation device while ablating the tissue adjacent to the pathway.

28. The method of claim 27, wherein the ablation device is an RF ablation device.

29. The method of claim 28, wherein the ablation device is a bipolar RF ablation device, or a monopole RF ablation device.

30. The method of claim 28, wherein the ablation device is a bipolar RF ablation device, the bipolar RF ablation device including a elongated member having a distal portion, a proximal portion and a middle portion between the distal and proximal portions, the distal portion including a first electrode and the middle portion including a second electrode, the distal portion being electrically isolated from the middle portion.

31. The method of claim 28 wherein the ablation device is a bipolar RF ablation device, the bipolar RF ablation device including a elongated member having a distal portion, the distal portion including a first electrode and the coring assembly including a second electrode.

32. The method of claim 21, further including treating the pathway with a therapeutic agent.
